(19) United States
(12) Patent Application Publication
(10) Pub. No.: US 2008/0020011 A1
(43) Pub. Date: Jan. 24, 2008

(54) THERAPEUTIC IMPLANT

(76) Inventors: Zvi Finkelstein, Asseret (IL); Dina Katsir, Beer Sheva (IL)

Correspondence Address: STITES & HARBISON PLLC, 1199 NORTH FAIRFAX STREET, SUITE 900, ALEXANDRIA, VA, 22314, US

(21) Appl. No.: 11/749,848

(22) Filed: May 17, 2007

Related U.S. Application Data
(63) Continuation-in-part of application No. PCT/IL05/01204, filed on Nov. 15, 2005.

(30) Foreign Application Priority Data
Nov. 19, 2004 (GB) 0425527.9

(51) Int. Cl. A61K 9/22; A61F 2/00; A61F 2/06; A61M 31/00; C07G 17/00; A61P 43/00; A61M 25/00; A61K 39/395

(52) U.S. Cl. 424/423; 424/138.1; 424/155.1; 435/267; 604/264; 604/288.04; 604/891.1; 623/1.11

(57) ABSTRACT

The invention relates an implant, which comprises a biologically compatible substrate, to which at least one moiety is bound by a ligand, the moiety being one which specifically binds to and selectively immobilizes, at least one known target present in body fluid. The moiety traps the target and thus enables undesired materials present in a body fluid to be destroyed locally in situ, or alternatively to be removed and destroyed ex vivo. A method for selectively removing from a mammalian body fluid in situ, at least one known target, also forms part of the invention.

The present patent application is a continuation-in-part of application no. PCT/IL/2005/001204.

### FIELD AND BACKGROUND OF THE INVENTION

The present invention relates to a method, an implant and use of specifically binding moieties, for selectively immobilizing and removing undesired targets from a mammalian body fluid in situ.

Antibiotics, radiotherapy and chemotherapy are widely used to treat the diseases which manifest themselves in body fluids, e.g., in the blood stream. Though being the most common methods, they suffer severe drawbacks, in particular high toxicity, damage to the healthy tissue including major organs, especially the heart, lung, liver, kidney, thyroid gland, bladder, reproductive organs and nervous system.

A lot of efforts have been invested to overcome these disadvantages, while eliminating undesired cells or tissue; see e.g. US Patent Application No. 2003097035 (Paulus, et al.), which describes a method for treating cancerous tissue in a patient, by positioning radioactive thermal seeds within the patient, each seed comprising a ferromagnetic core, a radioactive isotope and a palladium coating, and exposing the seeds to an oscillating magnetic field.

In U.S. Pat. No. 6,074,827 (Nelson, et al.) an integrated electrophoretic micro-device and method for nucleic acid purification and processing are described, and are preferably used to deplete or purge tumor cells or T lymphocytes from samples (e.g. from bone marrow), in order to make hematopoietic cell preparations for use in transplantation and in therapy. The device may include a bed of polymeric beads or paramagnetic beads or particles, coated with antibodies or other target-specific affinity binding moiety or e.g., streptavidin, coated for use with biotinylated antibodies. Another technique consists of coupling with radioactive atoms monoclonal antibodies against tumor antigens, whereby it is intended to limit the destructive power of radiation to those cells (cancerous) that have been “fingered” by the attached monoclonal antibody. Examples: Zevalin®. This is a monoclonal antibody against the CD20 molecule on B cells (and lymphomas) conjugated to either 1) the radioactive isotope indium-111 (¹¹¹In) or 2) the radioactive isotope yttrium-90 (⁹⁰Y) Both are given to the lymphoma patient, the ¹¹¹In version first followed by the ⁹⁰Y version (in each case supplemented with Rituxan). Bexxar® (Tositumomab). This is a conjugate of a monoclonal antibody against CD20 and the radioactive isotope iodine-131 (¹³¹I). It, too, is designed as a treatment for lymphoma. Although both Bexxar® and Zevalin® kill normal B cells, they don't harm the B-cell precursors because these do not express CD20. So, in time, the precursors can repopulate the body with healthy B cells.

U.S. Pat. No. 5,514,340 (Lansdorp, et al.) describes a device for separating magnetically labeled cells in a sample using an applied magnetic field, and methods of using the device to prepare purified cell preparations, preferably hematopoietic stem cell preparations depleted of selected cells such as T lymphocytes, tumor cells and/or red blood cells.

US Patent Application No. 20010023825 (Frumin, et al.) describes a method and device for moving, isolating and/or identifying particles in a sample by placing it in a spatially varying electrical field which follows a mathematical non-monotonous function, and may be applied to e.g. separation of malignant cells from healthy cells.

U.S. Pat. No. 6,268,119 (Sumita, et al.) describes a cell separation system comprising a porous structure of non-woven fabric constituting cell-capturing means, which permits passage of cells to be removed.

US Patent Application No. 20030231981 (Johnson, et al) describes a method for selectively binding and separating at least one component from whole blood or a body fluid, which is allowed to pass through a rigid integral separation matrix having a porous structure with a pore size ranging from 5 to 500 microns.

A method for the separation of cells from a mixed population of cells has been reported by Bigalow et al 1989, Journal of Immunological Methods 117: 289-293. These authors have reported the development of a hybrid of two separation methods, cellular adhesion chromatography (AC) and field-flow fractionation (FFF) that achieves effective separation of rat mesenteric B and T lymphocytes. This method combines the selective adhesion of AC and the control displacement forces of FFF, it also yields quantitative estimates of the binding forces of B and T lymphocytes to the adhesion surface of the system. This method uses an apparatus comprising two parallel glass plates and utilizes the different binding affinities to these plates by different cell types. This method has a major problem because it utilizes the inherent binding properties of cells to the particular glass surfaces and that it cannot be scaled up to separate a large number of cells.

U.S. Pat. No. 6,589,786 (Mangano, et al.) describes a device which enables discrete objects having a conducting inner core, surrounded by a dielectric membrane to be selectively inactivated by electric fields via irreversible breakdown of their dielectric membrane, and which may be applied to the selection, purification, and/or purging of desired or undesired biological cells from cell suspensions, without use of antibodies.

Use of Polymers for In Situ Therapy

Polymeric materials find increasing use in therapy. For example, US Patent Application 20020022013 (Leukel, et al.) relates to a biomedical molding comprising a non-biodegradable biocompatible organic polymer having specified radicals attached to its surface. The polymer may be, e.g., a polyurethane, epoxy resin, polyether, polyester, polyamide, polyimide, polyolefine, polybutadiene, polyisoprene, silicone, polysiloxane, perfluoroalkyl polyether, fluorinated poly-methacrylate, polyalkyl methacrylate, or a fluorinated polyolefine.

U.S. Pat. No. 5,800,516 (Fine et al.) describes a method for deployment and retrieval of a shape memory plastic tubular member, wherein, when having a first diameter, it is introduced to a treatment site, where it is expanded to a second diameter; when desired, it is retrieved from the site after causing it to return to its first diameter.

U.S. Pat. No. 5,474,563 (Myler et al) describes a retrievable elastomeric stent having proximal and distal engagement elements to allow an insertion/retrieval catheter to engage the stent for the application of axial force. When elongated in an axial direction, the stent is reduced in cross-sectional area, and it can then be removed.

U.S. Pat. No. 4,950,258 (Kawai, et al.) describes plastic molded articles for in situ applications, having characteristic properties of both shape-memory and biodegradability; they consist of homopolymers of lactide or glycolide or copolymers of lactide and glycolide.

U.S. Pat. No. 5,605,696 (Eury, et al.) describes drug loaded polymeric material in the form of a preferably porous intravascular stent. Specified polymers are polycaprolactone, poly(ethylene-co-vinyl acetate), poly(vinyl acetate), and silicone gum rubber, as well as non-degradable polymers, and biodegradable, bioabsorbable polymers such as poly-DL-lactic acid (DL-PLA), and poly-L-lactic acid (L-PLA), polyorthoesters, polyiminocarbonates, aliphatic polycarbonates, and polyphosphazenes.

U.S. Pat. No. 6,702,849 (Dutta, et al.) describes porous vascular grafts and stent covers formed of open-celled microcellular polymeric foams, and can be adapted for delivering therapeutic drugs in a blood vessel.

U.S. Pat. No. 5,800,828 (Dionne, et al.) describes an implantable biocompatible immunoisolatory vehicle, for delivery of therapeutic products including living cells. The vehicle may include a hydrogel such as alginate cross-linked with a multivalent ion.

U.S. Pat. No. 5,324,519 (Dunn et al.) and U.S. Pat. No. 6,395,293 (Polson et al.) describe compositions suitable for forming an in situ solid implant in an animal, from which implant biologically active agents may be released. US Patent Application No. 20040220296 (Lowman, et al.) describes a method of implanting a thermogelling hydrogel into a selected site of a mammal by injecting a hydrogel solution into a selected site whereby the hydrogel solidifies to form a solid implant at body temperature. The hydrogel solution may comprise poly(N-isopropyl acrylamide) and a second polymer, e.g. poly(ethylene glycol), poly(vinyl pyrrolidone) and poly(vinyl alcohol).

U.S. Pat. No. 6,514,688 (Muller-Schulte) describes the use of magnetic spherical cross-linked polyvinyl alcohol (PVAL) polymer particles for separating etc. biological materials. Example 15 gives experimental details for binding streptavidin to the matrix. Biotinylated DNA fragments can be bound to this matrix according to known methods.

Vein or artery catheters including polymeric components are widely in use in medical treatment as well as a diagnostic tool. For example, in U.S. Pat. No. 4,392,848 (Lucas et al.), at least part of an antimicrobial catheter comprises a permeable polymer, e.g. a silicone polymer such as polydimethylsiloxane. U.S. Pat. No. 5,470,307 (Lindall) describes a catheter having a therapeutic agent chemically bonded to a substrate on its exterior surface using a linker which photolytically releases the agent upon exposure to light energy; the substrate may include materials such as glass, polyamide, polyester, polyolefin, polypropylene, polyurethane, or latex.

Diagnostic implants including polymers are in use today for some assignments, for example to check on the blood flow, tracing blood clots, or to monitor concentration levels of a specific substrate, see e.g. US Patent Application No. 20040176672 (Silver et al.). which discloses a sensor for implantation within a blood vessel to monitor a substance in or property of blood, and which has a layer that minimizes the formation of thrombus and may comprises a hydrogel, e.g. poly(ethylene glycol), poly(N-vinyl pyrrolidone), or poly(hydroxyethylmethacrylate).

Treating polymer surfaces intended for contact with (e.g.) body fluids is also known. Thus, on U.S. Pat. No. 5,409,696 (Narayanan, et al), polymeric surfaces of medical devices or components thereof present an anti-thrombogenic, fibrinolytic or thrombolytic interface with body fluids such as blood during implantation or medical procedures. Suitable polymers are polyurethanes, and polyurethane-polyester, polyurethane-polyether and nylon-polyether copolymers, and silicone rubber. U.S. Pat. No. 6,638,728 (Desai et al.) describes a surface coating on e.g. polystyrene, consisting essentially of, in polymeric form, streptavidin, avidin or a deglycosylated avidin, wherein more than 50% of the polymer is a combination of dimers, trimers and tetramers of the native molecule; the products are said to have a high capacity for capturing target molecules, thus yielding assays with enhanced sensitivity. U.S. Pat. No. 5,795,719 (Vaslin et al.) describes latex microspheres obtained by polymerization of ethylenically unsaturated monomers, having surface functional groups including grafted biotinyl residues, and corresponding avidin- or streptavidin-biotin complexes, as agents for diagnosis, or biological or immunological assays. Antibodies. Antibodies are commonly used for biological assays, therapy and diagnosis, and for this purpose they are attached to a suitable substrate, especially polymers. Some constructs including antibodies have already been mentioned above. Additionally, e.g., in U.S. Pat. No. 4,582,810 (Rosenstein), a suspension of diagnostic particles comprising antibody molecules attached to a carboxylate derivatized polymer core (e.g. polystyrene or polyacrylamide) is provided for agglutination tests. The antibody is linked to the core through an avidin-biotin bridge. Avidin is joined by an amide bond to carboxyl groups on the core, and biotin is linked by an amide bond to amino groups on the antibody molecule.

The use of antibodies in the living body is usually referred as immunotherapy when designated for therapy or immuno- diagnosis which is in use for research and diagnosis, both of which are common tools in use today. Thus, e.g., U.S. Pat. No. 6,455,043 (Grillo-Lopez) treatment of B-cell lymphomas is disclosed, and in particular administration of anti-CD20 antibodies to patients having non-Hodgkins lymphomas. Similarly, U.S. Pat. No. 5,736,137 (Anderson et al.) describes therapeutic strategies which include the use of administration of immunologically active mouse/human chimeric anti-CD20 antibodies, radiolabeled anti-CD20 antibodies, while US Patent Application No. 2003219433 (Hansen et al.) describes humanized, chimeric and human anti-CD20 antibodies and CD 20 antibody fusion proteins that bind to CD20, which are thus useful for treatment and diagnosis of B-cell disorders, and autoimmune diseases.

U.S. Pat. No. 6,264,596 (Weadock) describes a radioactive device formed in situ and adapted for placement at an intravascular treatment site to inhibit restenosis, having a first substance (A) immobilized on said device surface, and adapted to selectively bind a radioactive second substance (B) when this is intravascularly injected. In specified pairs of (A) and (B), (A) is avidin, streptavidin, or a protein, and (B) is radio-labeled biotin, or radio-labeled monoclonal or polyclonal antibodies; (A) is protamine and (B) is radio-labeled heparin; (A) is a protein and (B) is radio-labeled antibody having an affinity for said protein; (A) biotin and said (B) is radio-labeled avidin or streptavidin.

U.S. Pat. No. 5,667,523 (Bynon, et al.) discloses a dual supported intra-luminal graft comprising a biocompatible flexible layer sandwiched between two structural support layers, e.g. a first stent is concentrically retained within a tubular shaped PTFE graft which is concentrically retained within a second stent.

The entire contents of the patents and published patent applications mentioned in the present specification are incorporated herein by reference.

Notwithstanding the extensive efforts described in the prior art, there is an acute need for a therapeutic method which can provide an effective elimination of pathogenic factors from whole blood, while minimizing the damage caused to healthy tissue and organs.

### SUMMARY OF THE INVENTION

The present invention accordingly provides in one aspect, an implant, adapted for insertion in a mammalian body cavity, for use in selectively removing from a body fluid and immobilizing on the implant, at least one known target selected from pathogenic factors, antigens and antigenic determinants, including cells and cell fragments which are at least in part cancerous or pathogenically infected, wherein the implant comprises a surface layer including at least one moiety selected from antibodies and fragments thereof, which specifically bind to the at least one known target, and wherein the implant comprises a biologically compatible porous or non-porous substrate, to which the at least one moiety is bound by a ligand in the surface layer. In a modification, the target may include Implant-rejecting cells and cells having defective autoimmune properties.

In another aspect, the present invention provides a method for selectively removing from a mammalian body fluid in situ, at least one known target, which comprises the following steps (A) and either (B) or (C), namely:
(A) exposing to the body fluid in a cavity of the mammalian body, an implant inserted in the cavity, wherein the implant comprises a surface layer including at least one moiety which specifically binds to and thus immobilizes the at least one known target, and wherein the implant comprises a biologically compatible porous or non-porous substrate, to which the at least one moiety is bound by a ligand in the surface layer; and, after a predetermined time interval,
either (B) removing from the mammalian body cavity, the implant including the at least one target bound to the at least one moiety, and in an optional further step, destroying ex-vivo the at least one target bound to the at least one moiety; or (C) destroying in situ the at least one target bound to the at least one moiety.

In yet another aspect, the invention provides an implant for use in selectively removing from a mammalian body fluid and immobilizing on the implant, at least one known target selected from pathogenic factors, antigens and antigenic determinants, including cells and cell fragments which are at least in part cancerous or pathogenically infected, wherein the implant comprises a surface layer including at least one moiety selected from antibodies and fragments thereof, which specifically bind to the at least one known target, and comprises also a biologically compatible substrate, to which the at least one moiety is bound by a ligand in the surface layer, the implant being either adapted for suspension within the internal hollow space of an intra-luminal stent, or being suspended within the internal hollow space of an intra-luminal stent prior to insertion of the stent together with its suspended implant in the mammalian body.

In still another aspect, the invention provides a method for selectively removing from a mammalian body fluid in situ, at least one known target, which comprises the following steps (A) and either (B) or (C), namely:
(A) exposing to the body fluid in the internal hollow space of an intra-luminal stent, an implant suspended in the internal hollow space, wherein the implant comprises a surface layer including at least one moiety which specifically binds to and thus immobilizes the at least one known target, and wherein the implant comprises a biologically compatible substrate, to which the at least one moiety is bound by a ligand in the surface layer; and, after a predetermined time interval,
either (B) removing from the mammalian body the implant either with or without the intra-luminal stent, the implant including the at least one target which is bound to the at least one moiety, and in an optional further step, destroying ex-vivo the at least one target bound to the at least one moiety;
or (C) destroying in situ the at least one target bound to the at least one moiety.

In a further aspect, the invention relates to an essentially multi-part intra-luminal device, adapted for insertion into and retrieval from a mammalian body cavity, which comprises a combination of at least two concentric tubes separated by spacers, wherein at least one of said tubes is an implant as defined herein, and at least one other of said tubes carries a therapeutically active substance, such as may be selected from chemotherapeutic compounds and a sealed radiation source.

In yet a further aspect, the invention relates to use of at least one moiety selected from antibodies and fragments thereof, which specifically bind to at least one known target present in body fluid, wherein the target is selected from pathogenic factors, antigens and antigenic determinants, including cells and cell fragments which are at least in part cancerous or pathogenically infected, in the manufacture of a medicament, for treating diseases associated with the at least one known target, and which includes an implant for selectively removing from the body fluid and immobilizing the at least one known target on the implant, and wherein the implant comprises a biologically compatible porous or non-porous substrate, to which the at least one moiety is bound by a ligand, the implant being either adapted for suspension within the internal hollow space of an intra-luminal stent, or being suspended within the internal hollow space of an intra-luminal stent prior to insertion of the stent together with its suspended implant in the mammalian body.

### BRIEF DESCRIPTION OF THE DRAWINGS

FIG. 1 illustrates an embodiment of the implant of the invention.

FIG. 2 illustrates a further embodiment of the implant of the invention.

### DEFINITIONS

The implant of the present invention, whether or not it is adapted for insertion within the internal hollow space of an intra-luminal stent, is one which is capable of being manufactured and stored ex-vivo, and thus excludes similar constructs which are formed in vivo exclusively.

The target, which in accordance with the present invention is removed from body fluids and immobilized on the inventive implant, is one which is either naturally present in the body fluids, or is present due to a disease or condition of the patient, and thus excludes biological or chemical materials which hare been artificially injected into the body of a patient.

As stated above, in a particular aspect of the present invention, the implant may be either adapted for suspension within the internal hollow space of an intra-luminal stent, or is suspended within the internal hollow space of an intra-luminal stent prior to insertion of the stent together with its suspended implant in the mammalian body. The manner of insertion of intra-luminal stents into body cavities is well known and per se does not form part of the present invention. However, the implant may be adapted for suspension by including in the suitably configured substrate, possibly retractable stops or spacers capable of holding the implant rigidly within the internal hollow space of an intra-luminal a stent. In an alternative embodiment, a stent may be configured with the necessary holding elements. In still another embodiment, the inner surface of a stent and implant may both be configured, possibly with mutually cooperating elements, in order to achieve the object of holding the implant rigidly in its desired location, until removal is desired. Unless there are medical reasons for maintaining the stent itself in the intra-luminal space, withdrawal therefrom of the implant may be effected either with or without the attached stent. In another mode of operation, the implant and(or) the stent may be constructed of physiologically degradable materials which will be decomposed into harmless components, after a predetermined time lapse.

When the implant is adapted for suspension within the internal hollow space of an intra-luminal stent, it may have a variety of configurations. For example it may be in the form of a hollow cylinder, so that in operation it will be like “a stent within a stent”. Alternative configurations, such as an open helix, or a zig-zag concertina like mode, will suggest themselves to persons of the art, and all such configurations come within the scope of the present invention. Of course, it may well be necessary when selecting a configuration for this aspect of the inventive implant, to present the greatest possible surface area to the flow of blood, without however unduly restricting this flow.

### DETAILED DESCRIPTION OF THE INVENTION

The implant of the invention may be radioactive or non-radioactive, and is preferably further characterized by at least one of the following features:
(a) the at least one target is selected from entities comprising antigens and antigenic determinants, and the at least one moiety is selected from monoclonal antibodies which are specific for the at least one target;
(b) it includes a device for connection to a catheter;
(c) it includes, and is adapted for slow release of, at least one pharmacologically active compound selected from the group consisting of antibacterial drugs, anti-fungal drugs, anti-neoplastic drugs, anti-thrombotic drugs, anti-toxin drugs and antiviral drugs.

The ligand referred to above preferably comprises at least one substance selected from avidin, biotin, streptavidin, and their analogues.

The specifically binding moiety referred to herein may e.g. be selected from proteins, polypeptide or fragments thereof, antibodies or fragments thereof, carbohydrates (including polysaccharides), hormones, antioxidants, glycoproteins, lipoproteins, lipids, fat soluble vitamins, bile acids, reactive dyes, allantoin, uric acid, polymyxin, nucleic acid molecules (DNA, RNA, single stranded, double stranded, triple stranded or combinations thereof), or combinations thereof.

In a particular embodiment, the moiety is selected from monoclonal antibodies, polyclonal antibodies, synthetic antibodies, antigenic affinity synthetic fragments, antibody fragments retaining their antigenic affinity, Fv antibody fragments, radio labelled antibodies and biotinylated antibodies. By way of illustrative example only, when the moieties are monoclonal antibodies, they may be such that they bind specifically to B- or T-cell antigenic determinants, e.g., the CD-20 antigenic determinant of B-cells for non-Hodgkins lymphoma therapy, anti-CD20 or anti-CD52 as a therapy for chronic lymphocytic leukemia, anti-CD52 as a therapy for polymphocytic leukemia, anti-CD22 or anti-CD52 as a therapy for hairy cell leukemia, or anti-CTLA-4 as a therapy for acute leukemia therapy.

It will be appreciated that antiviral drugs and antiviral antibodies may be applied separately or in combination.

The method of the invention is preferably further characterized by at least one of the following features:
(a) the at least one target is selected from entities comprising antigens and antigenic determinants, and the at least one moiety is selected from monoclonal antibodies which are specific for the at least one target;
(b) the implant is connectable to a catheter;
(c) the implant includes, and is adapted for slow release of, at least one pharmacologically active compound selected from the group consisting of antibacterial drugs, anti-fungal drugs, anti-neoplastic drugs, anti-thrombotic drugs, anti-toxin drugs and antiviral drugs;
(d) the body cavity is a blood vessel and the body fluid is blood;
(e) the at least one target is selected from a population of cells and cell fragments, which is at least in part cancerous or pathogenically infected;
(f) said optional further step and said step (C) are carried out by locally applying to said at least one target which is bound to said at least one moiety, at least one of the following, namely, radiation, heat (hyperthermia), sonication, immunotherapy, radioimmunotherapy, genetic therapy or controlled drug release;
(g) the specific monoclonal antibodies in the surface layer are renewed and(or) supplemented by direct administration to the mammalian body of monoclonal antibodies adapted for specific binding to the implant as well as to said target.

In paragraph (f), above, application of radiation may be by any known technique, e.g., from an external radiation device, or by radioactive drug administration, or in situ radiation.

Brachytherapy is a form of radiotherapy where a sealed radioactive source is placed inside or next to the area requiring treatment. It will be appreciated that the present invention may be adapted to this technique, e.g. as follows. An implant (such as a stent) which comprises a surface layer including at least one moiety selected from antibodies and fragments thereof, which specifically binds to a desired target known to be amenable to destruction by radiation, may be inserted into a blood vessel. At an optimum, possibly predetermined time thereafter, a second implant, e.g. of stent-like configuration, designed to “dock” with the first implant, in the inner space of the latter, is then inserted. The second implant carries a sealed radioactive source. At least the second implant is designed to be readily retrievable. In an alternative embodiment, the combination of first and second implants is designed to be readily retrievable. In this manner, the target accumulates on the first implant and is destroyed by the radiation from the second implant, while the radioactive source may be withdrawn when desired.

In a analogous embodiment, in place of (or in addition to) the sealed reactive source, there may be used a releasable chemotherapeutic agent, effective to act against a desired target being collected on the surface of the first implant.

It will be appreciated that the present device may include utilization for example of polymeric beads and other particles (as the substrate) to which streptavidin and biotinylated antibodies may be attached sequentially, similarly to U.S. Pat. No. 6,074,827, and see also U.S. Pat. No. 6,514,688, as well as U.S. Pat. No. 5,795,719 which relates to latex microspheres having surface functional groups pertinent to the present invention, and US 4,582,810 (which describes particles comprising antibodies attached to a polymer via an avidin-biotin bridge); all of these could find application in the present invention.

It is apparent that, in contrast to known ex-vivo use, in the present instance such beads or particles would have to be safely restrained within a suitable membrane, which would be permeable only to circulating body fluids and their contents.

When the substrate used in the present device is polymeric, this may be constructed from any polymer known to be biocompatible, and generally non-biodegradable, e.g. as mentioned in US 20020022013 (see above). The polymeric substrate would have to be capable of a surface reaction to attach antibodies, in the general case via one or more ligands. It is contemplated that the device, in effect the substrate, may have any shape compatible with the purpose of being in semi-permanent contact with body fluids in situ, e.g., in a blood vessel; exemplary shapes are: a solid-surfaced or perforated hollow tube, a tube constructed from hollow rings connected by ribs, or a disc, rod, ring, helix or sphere.

It is believed that while polymeric substrates for present use will be generally non-biodegradable, nevertheless for some applications biodegradability may be a useful property, and consequently biodegradable polymers as described e.g. in the above-mentioned U.S. Pat. No. 4,950,258, could be useful in the present context, subject of course to their ability to undergo a surface reaction to attach ligands and(or) antibodies, as mentioned before.

It is presently contemplated that, notwithstanding the fact that the present device is principally concerned with the capture of adverse circulating entities in body fluids, rather than release (except as an optional additional feature), nevertheless for reasons of biocompatibility, known devices for in situ use for releasing therapeutic substances into the body, could be useful also as substrates for the present device, and in this connection, the devices of the above-mentioned U.S. Pat. No. 5,605,696, U.S. Pat. No. 6,702,849 and U.S. Pat. No. 5,800,828, could be particularly relevant.

The implant of the present invention could also be formed in situ, e.g. as described in above mentioned U.S. Pat. No. 5,324,519 and U.S. Pat. No. 6,395,293.

As mentioned elsewhere, the device, after having trapped the adverse antigenic entities, may be suitably treated locally in situ, on order to destroy such entities. In the alternative, if a retrievable device has been employed, this may be recovered and treated ex-vivo. Analogous retrievable bodies are known in the art, see e.g. the above-mentioned U.S. Pat. No. 5,800,516 and U.S. Pat. No. 5,474,563, and it is contemplated that such bodies may be adapted for use in the present invention.

Similarly, diagnostic implants including polymers, as described e.g. in US 20040176672, as well as surface-treated polymers intended for contact with (e.g.) body fluids, as e.g. in U.S. Pat. No. 5,409,696 and U.S. Pat. No. 6,638,728, could be useful as substrates for the present device.

The possibility is also contemplated of adapting the catheter of e.g., U.S. Pat. No. 5,470,307 (which describes a catheter having a therapeutic agent chemically bonded to a substrate on its exterior surface), for the purposes of the present invention.

### DESCRIPTION OF PRESENTLY PREFERRED EMBODIMENTS

Mature B-cells are characterized by antigenic determinants which are highly specific molecules binding to specific antibodies. CD-20 is such an antigenic determinant, presented on the outer surface of B-cells, both normal mature B-cells and malignant B-cells.

Non-Hodgkins Lymphoma is a common cancer developed in B and T cells; 53,000 people have been diagnosed with this type of Lymphoma in the year 2003, in the U.S.A. alone.

Non-Hodgkins Lymphoma is generally characterized by an abnormal proliferation of the B-cell lymphocytes, which in addition to the fact that it is a life threatening situation, causes the following symptoms: anemia, swollen lymph nodes, weakness and sickness. The therapy currently used is a combination of chemotherapy and radiotherapy according to the progressive state of the disease. Radiotherapy focuses on some of the major lymph nodes, which contain concentrated population sites of the white blood cells including all types of lymphocytes. Scientific progress based on the vast research conducted in this field, is providing progressively more accurate treatment, while lowering the dosages involved. However, radiotherapy, while attacking the cancerous cells in the lymph nodes, also destroys many other types of blood cells which populate the lymph nodes as well. This major disadvantage disrupts the immune system and destroys its major components. Moreover, radiotherapy attacks the mature cells as well as the young cells not yet finally differentiated. Even if the disease situation deteriorates, not all lymph nodes can be radiated simultaneously; to do so would make the body susceptible to life threatening disease because of breakdown of the immune system.

The present invention includes an implant which is adapted inter alia to immobilize a specific cell population, including infected cells, such as B-cells, in circulating blood or e.g. in circulating lymphatic fluids. The device is fixed in a predetermined easily traced location. These features provide a concentrated B-cell population site. The device isolates a considerable part of B-cells from whole blood. The device including the antibodies may be referred to as “the trap area”.

On radiating this trap, the bound B-cells are eliminated, thus providing a much more specific radiotherapy than the one in use today in cancer clinics and hospitals. The device has a surface layer which includes CD-20 antibodies, preferably biotinylated antibodies containing a biotin-avidin link, so as to provide a high binding affinity option for specific cell binding and isolation, and a substrate, particularly a polymeric substrate, which contains streptavidin molecules and these CD-20 antibodies.

It is presently contemplated that the inventive implant and method could also be used to at least substantially reduce the population of metastasized cells and to inhibit the proliferation activity of certain cells.

The present device may be designed to be connected to a catheter, and inserted into a small artery or a large vein. It is preferably made of a non-degradable polymer, to which may be attached the CD-20 antibodies, preferably in the manner just recited.

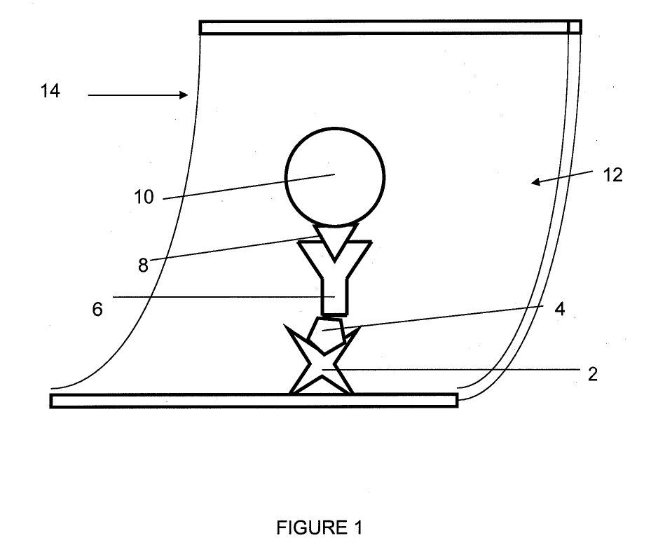

An illustrative schematic embodiment of the implant of the invention is shown in FIG. 1, in which none of the referenced features are drawn to scale. The illustrative device shows the inner wall 12 of stent-like device 14 (part of the circumferential wall of which is shown for illustrative purposes as cut open) which may be fabricated from e.g. a suitable polymeric material. Attached to inner wall 12, to be exposed to a body fluid such as circulating blood or lymphatic fluid, are many monoclonal antibodies which are attached in the manner illustrated (very greatly enlarged) in the case of a representative single antibody 6. Antibody 6 is attached to wall 12 via a ligand constituted by biotin 4 and avidin or streptavidin 2. The elements 14-2-4-6 in combination (which are preferably attached one to another by covalent bonds), thus represent an illustrative embodiment of the implant of the invention. The monoclonal antibody 6 in this illustration, is specific for antigenic determinant 8 on the outer surface of cell 10, and thus captures it as shown in FIG. 1.

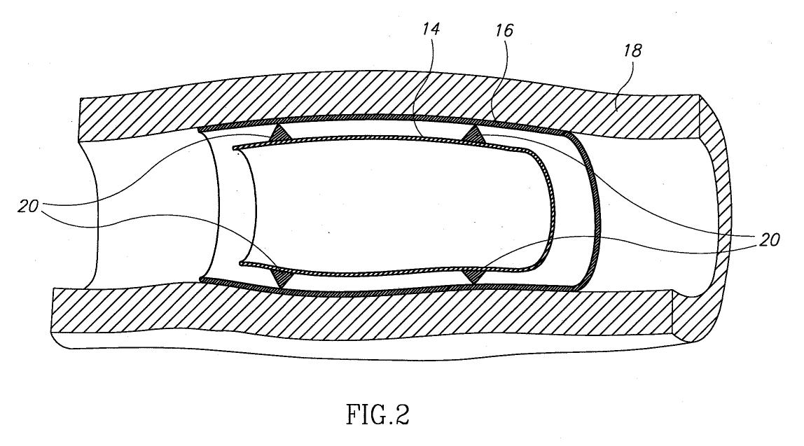

In another particular embodiment of the inventive implant, the stent-like device 14 shown in FIG. 1 may be suspended and supported within a regular stent 16 (which fits the blood vessel wall 18) by means of spacers 20 (see FIG. 2, showing items 14, 16 and 20 in cut-away format). The thus-formed device comprising two concentric tubes may be assembled ex vivo and subsequently implanted in a blood vessel. Alternatively, stent 16 may be first inserted in the blood vessel (or may already exist in situ), followed by tube 14 with integral spacers 20. Components 14, 16 and 20 may be biodegradable or non-biodegradable, as desired. The device illustrated in FIG. 2 may be arranged for withdrawal of the whole simultaneously, or for the withdrawal only of tube 14 with integral spacers 20. In alternative embodiments, spacers 20 may be integral with stent 16 only, or some may be integral with stent 16 and some with tube 14.

### The Invention Will Be Illustrated by the Following Non-Limiting Example.

### EXAMPLE

Anti-CD20 antibody solution 0.25 mg/ml in PSA (Pig Serum Albumin) 10% solution at pH 7.8 is reacted with an equivalent amount of itaconic anhydride and then the product is copolymerized with approximately 20× its weight of N-isopropylacrylamide. The conjugated itaconic residue thus functions as ligand and also forms part of the substrate. The resulting copolymer was formed into a tube 10 cm×5 mm, by blending with a second polymer, e.g. poly(ethylene glycol), poly(vinyl pyrrolidone) or(and) poly(vinyl alcohol, and if necessary heating to promote gelation. In an alternative procedure, one or more monomers corresponding to the second polymer may participate in the copolymerization reaction.

The tube is carefully washed in saline 0.9% (NaCl). The tube is inserted using a catheter to a pig vein for 24 hours. Blood samples are taken every 30 min and a WBC blood count is taken to monitor inflammation. The number of B-cells in the blood is evaluated using FACS analysis. After this time period, the catheter may be removed.

### ADVANTAGES OF THE INVENTION

This invention offers a safe method for therapy, based on technologies already approved, replacing the dangerous total radiation therapy technique and thus opens up an entirely new outlook on therapy. In particular, radiation treatment is in the present case focused on a localized area of the body, desirably in a preselected area remote from organs susceptible to radiation damage, instead of the more hazardous approach of radiating lymph node areas in a broad sense, or may even be carried out, in the alternative, ex vivo. Thus, it id believed that total recovery prospects for the patient are greatly improved.

Our new technology specifically targets, e.g., a mature cell population exclusively, including malignant cells and imposes only minimal harm on other cells. This type of treatment can lower the stress imposed unnecessarily upon the whole WBC (White Blood Cells) population.

Other advantages include minimal side effects including decreased tissue damage and inflammation, and little or no immune suppression or other harm to the immune system, increased efficiency and relatively low cost of treatment—including relative simplicity in operation.

While the invention has been described with respect to specific embodiments including presently preferred modes of carrying out the invention, those skilled in the art will appreciate that there are numerous possibilities of variations and permutations that nevertheless fall within the spirit and scope of the invention.

1. An implant, adapted for insertion in a mammalian body cavity, for use in selectively removing from a body fluid and immobilizing on said implant, at least one known target selected from pathogenic factors, antigens and antigenic determinants, including cells and cell fragments which are at least in part cancerous or pathogenically infected, wherein said implant comprises a surface layer including at least one moiety selected from antibodies and fragments thereof, which specifically bind to said at least one known target, and wherein said implant comprises a biologically compatible substrate, to which said at least one moiety is bound by a ligand in said surface layer.

2. Implant according to claim 1, wherein said moiety is selected from
monoclonal antibodies, polyclonal antibodies, synthetic antibodies, antigenic affinity synthetic fragments, antibody fragments retaining their antigenic affinity, Fv antibody fragments, radio labelled antibodies and biotinylated antibodies.

3. Implant according to claim 1, which is further characterized by at least one of the following features:
(a) said at least one target is selected from entities comprising antigens and antigenic determinants, and said at least one moiety is selected from monoclonal antibodies which are specific for said at least one target;
(b) it includes a device for connection to a catheter;
(c) it includes, and is adapted for slow release of, at least one pharmacologically active compound selected from the group consisting of antibacterial drugs, anti-fungal drugs, anti-neoplastic drugs, anti-thrombotic drugs, anti-toxin drugs and antiviral drugs.

4. Implant according to claim 3, wherein said substrate is selected from natural and synthetic polymers, ceramics, glass, metals, metal oxides and fabrics.

5. Implant according to claim 4, which is further characterized by at least one of the following features:
said ligand comprises at least one substance selected from avidin, biotin, streptavidin, and their analogues;
said monoclonal antibodies bind specifically to B- or T-cell antigenic determinants.

6. A method for selectively removing from a mammalian body fluid in situ, at least one known target, which comprises the following steps (A) and either (B) or (C), namely:
(A) exposing to said body fluid in a cavity of said mammalian body, an implant inserted in said cavity, wherein said implant comprises a surface layer including at least one moiety which specifically binds to and thus immobilizes said at least one known target, and wherein said implant comprises a biologically compatible substrate, to which said at least one moiety is bound by a ligand in said surface layer; and, after a predetermined time interval,
either (B) removing from said mammalian body cavity, said implant including said at least one target which is bound to said at least one moiety, and in an optional further step, destroying ex-vivo said at least one target bound to said at least one moiety;
or (C) destroying in situ said at least one target bound to said at least one moiety.

7. Method according to claim 6, wherein said moiety is selected from proteins, polypeptide or fragments thereof, antibodies or fragments thereof, carbohydrates (including polysaccharides), hormones, antioxidants, glycoproteins, lipoproteins, lipids, fat soluble vitamins, bile acids, reactive dyes, allantoin, uric acid, polymyxin, nucleic acid molecules (DNA, RNA, single stranded, double stranded, triple stranded or combinations thereof), or combinations thereof.

8. Method according to claim 6, wherein said moiety is selected from monoclonal antibodies, polyclonal antibodies, synthetic antibodies, antigenic affinity synthetic fragments, antibody fragments retaining their antigenic affinity, Fv antibody fragments, radio labelled antibodies and biotinylated antibodies.

9. Method according to claim 6, which is further characterized by at least one of the following features:
(a) said at least one target is selected from entities comprising antigens and antigenic determinants, and said at least one moiety is selected from monoclonal antibodies which are specific for said at least one target;
(b) said implant is connectable to a catheter;
(c) said implant includes, and is adapted for slow release of, at least one pharmacologically active compound selected from the group consisting of antibacterial drugs, anti-fungal drugs, anti-neoplastic drugs, anti-thrombotic drugs, anti-toxin drugs and antiviral drugs;
(d) said body cavity is a blood vessel and said body fluid is blood;
(e) said at least one target is selected from a population of cells and cell fragments, which is at least in part cancerous or pathogenically infected;
(f) said optional further step and said step (C) are carried out by locally applying to said at least one target which is bound to said at least one moiety, at least one of the following, namely, radiation, heat (hyperthermia), sonication, immunotherapy, radioimmunotherapy, genetic therapy or controlled drug release;
(g) the specific monoclonal antibodies in the surface layer are renewed and(or) supplemented by direct administration to the mammalian body of monoclonal antibodies adapted for specific binding to the implant as well as to said target.

10. Method according to claim 9, which is further characterized by at least one of the following features:
(i) said substrate is selected from natural and synthetic polymers, ceramics, glass, metals, metal oxides and fabrics;
(ii) said at least one target is selected from cancer-affected cells such as mature B-cells and T-cells.
(iii) said directly administered monoclonal antibodies comprise a bound ligand.

11. Method according to claim 10, which is further characterized by at least one of the following features:
said ligand comprises at least one substance selected from avidin, biotin, streptavidin, and their analogues;
said monoclonal antibodies bind specifically to B- or T-cell antigenic determinants.

12. An implant for use in selectively removing from a mammalian body fluid and immobilizing on said implant, at least one known target selected from pathogenic factors, antigens and antigenic determinants, including cells and cell fragments which are at least in part cancerous or pathogenically infected, wherein said implant comprises a surface layer including at least one moiety selected from antibodies and fragments thereof, which specifically bind to said at least one known target, and comprises also a biologically compatible substrate, to which said at least one moiety is bound by a ligand in said surface layer, said implant being either adapted for suspension within the internal hollow space of an intra-luminal stent, or being suspended within the internal hollow space of an intra-luminal stent prior to insertion of the stent together with its suspended implant in the mammalian body.

13. Implant according to claim 12, wherein said moiety is selected from monoclonal antibodies, polyclonal antibodies, synthetic antibodies, antigenic affinity synthetic fragments, antibody fragments retaining their antigenic affinity, Fv antibody fragments, radio labelled antibodies and biotinylated antibodies.

14. Implant according to claim 12, which is further characterized by at least one of the following features:
(a) said at least one target is selected from entities comprising antigens and antigenic determinants, and said at least one moiety is selected from monoclonal antibodies which are specific for said at least one target;
(b) it includes a device for connection to a catheter;
(c) it includes, and is adapted for slow release of, at least one pharmacologically active compound selected from the group consisting of antibacterial drugs, antifungal drugs, anti-neoplastic drugs, anti-thrombotic drugs, anti-toxin drugs and antiviral drugs.

15. Implant according to claim 14, wherein said substrate is selected from natural and synthetic polymers, ceramics, glass, metals, metal oxides and fabrics.

16. Implant according to claim 15, which is further characterized by at least one of the following features:
said ligand comprises at least one substance selected from avidin, biotin, streptavidin, and their analogues;
said monoclonal antibodies bind specifically to B- or T-cell antigenic determinants.

17. A method for selectively removing from a mammalian body fluid in situ, at least one known target, which comprises the following steps (A) and either (B) or (C), namely:
(A) exposing to said body fluid in the internal hollow space of an intra-luminal stent, an implant suspended in said internal hollow space, wherein said implant comprises a surface layer including at least one moiety which specifically binds to and thus immobilizes said at least one known target, and wherein said implant comprises a biologically compatible substrate, to which said at least one moiety is bound by a ligand in said surface layer; and, after a predetermined time interval,
either (B) removing from said mammalian body said implant either with or without said intra-luminal stent, said implant including said at least one target which is bound to said at least one moiety, and in an optional further step, destroying ex-vivo said at least one target bound to said at least one moiety;
or (C) destroying in situ said at least one target bound to said at least one moiety.

18. Method according to claim 17, wherein said moiety is selected from proteins, polypeptide or fragments thereof, antibodies or fragments thereof, carbohydrates (including polysaccharides), hormones, antioxidants, glycoproteins, lipoproteins, lipids, fat soluble vitamins, bile acids, reactive dyes, allantoin, uric acid, polymyxin, nucleic acid molecules (DNA, RNA, single stranded, double stranded, triple stranded or combinations thereof), or combinations thereof.

19. Method according to claim 17, wherein said moiety is selected from monoclonal antibodies, polyclonal antibodies, synthetic antibodies, antigenic affinity synthetic fragments, antibody fragments retaining their antigenic affinity, Fv antibody fragments, radio labelled antibodies and biotinylated antibodies.

20. Method according to claim 17, which is further characterized by at least one of the following features:
(a) said at least one target is selected from entities comprising antigens and antigenic determinants, and said at least one moiety is selected from monoclonal antibodies which are specific for said at least one target;
(b) said implant is connectable to a catheter;
(c) said implant includes, and is adapted for slow release of, at least one pharmacologically active compound selected from the group consisting of antibacterial drugs, anti-fungal drugs, anti-neoplastic drugs, anti-thrombotic drugs, anti-toxin drugs and antiviral drugs;
(d) said body fluid is blood;
(e) said at least one target is selected from a population of cells and cell fragments, which is at least in part cancerous or pathogenically infected;
(f) said optional further step and said step (C) are carried out by locally applying to said at least one target which is bound to said at least one moiety, at least one of the following, namely, radiation, heat (hyperthermia), sonication, immunotherapy, radioimmunotherapy, genetic therapy or controlled drug release;
(g) the specific monoclonal antibodies in the surface layer are renewed and(or) supplemented by direct administration to the mammalian body of monoclonal antibodies adapted for specific binding to the implant as well as to said target.

21. Method according to claim 20, which is further characterized by at least one of the following features:
(i) said substrate is selected from natural and synthetic polymers, ceramics, glass, metals, metal oxides and fabrics;
(ii) said at least one target is selected from cancer-affected cells such as mature B-cells and T-cells.
(iii) said directly administered monoclonal antibodies comprise a bound ligand.

22. Method according to claim 21, which is further characterized by at least one of the following features:
said ligand comprises at least one substance selected from avidin, biotin, streptavidin, and their analogues;
said monoclonal antibodies bind specifically to B- or T-cell antigenic determinants.

23. An essentially multi-part intra-luminal device, adapted for insertion into and retrieval from a mammalian body cavity, which comprises a combination of at least two concentric tubes separated by spacers, wherein at least one of said tubes is an implant as defined in claim 1, and at least one other of said tubes carries a therapeutically active substance selected from chemotherapeutic compounds and a sealed radiation source.

24. A device according to claim 23, wherein in said implant, said moiety is selected from monoclonal antibodies, polyclonal antibodies, synthetic antibodies, antigenic affinity synthetic fragments, antibody fragments retaining their antigenic affinity, Fv antibody fragments, radio labelled antibodies and biotinylated antibodies.

25-27. (canceled)

28. A device according to claim 23, wherein said implant is further characterized by at least one of the following features:
(a) said at least one target is selected from entities comprising antigens and antigenic determinants, and said at least one moiety is selected from monoclonal antibodies which are specific for said at least one target;
(b) it includes a device for connection to a catheter;
(c) it includes, and is adapted for slow release of, at least one pharmacologically active compound selected from the group consisting of antibacterial drugs, anti-fungal drugs, anti-neoplastic drugs, anti-thrombotic drugs, anti-toxin drugs and antiviral drug;.
(d) said substrate is selected from natural and synthetic polymers, ceramics, glass, metals, metal oxides and fabrics;
(e) said ligand comprises at least one substance selected from avidin, biotin, streptavidin, and their analogues;
(f) said monoclonal antibodies bind specifically to B- or T-cell antigenic determinants.
